(19) United States
(12) Patent Application Publication
(10) Pub. No.: US 2011/0218460 A1
(43) Pub. Date: Sep. 8, 2011

(54) FALL DETECTING DEVICE AND FALL DETECTING METHOD

(75) Inventor: Toshihiro Masuzawa, Shiojiri-shi (JP)

(73) Assignee: SEIKO EPSON CORPORATION, Shinjuku-ku (JP)

(21) Appl. No.: 13/043,381

(22) Filed: Mar. 8, 2011

(30) Foreign Application Priority Data
Mar. 8, 2010 (JP) 2010-050232

(51) Int. Cl. A61B 5/11

(52) U.S. Cl. 600/595

(57) ABSTRACT

A fall detecting device includes: a detector unit that includes a plurality of tilt sensors each including a pair of electrodes disposed to oppose each other with the positional relationship therebetween being fixed and a conductor capable of freely moving between the pair of electrodes and changing an electrical-connection state between the pair of electrodes by means of the movement of the conductor resulting from a variation in posture of the pair of electrodes, wherein the plurality of tilt sensors are arranged so that directions in which the pairs of electrodes are opposed to each other are perpendicular to each other; a multivalued data output unit that acquires the electrical-connection states between the pair of electrodes of the plurality of tilt sensors and converts the acquired electrical-connection state into multivalued data on the basis of the ratios of the electrical-connection states in a predetermined first period.

### BACKGROUND

1. Technical Field

The present invention relates to a fall detecting device and a fall detecting method.

2. Related Art

A method in which a tilt sensor, an output state of which varies depending on a tilt angle, is attached to a person's body to detect that a solitary elderly person or a construction worker working alone has fallen has been known. For example, JP-A-2008-242704 has proposed a safety confirmation system including an information sending device collecting and sending information detected by the use of a tilt sensor and an information transmitting unit, where a solitary elderly person is assumed to be the wearer wearing the tilt sensor.

In JP-A-2008-242704, the output state of ON or OFF of the tilt sensor is detected as a signal at a predetermined interval and the occurrence of a fall is determined when the period during which the output state of the tilt sensor is OFF is long.

However, in the method described in JP-A-2008-242704, the occurrence of a fall is determined when the period during which the tilt sensor has a horizontal posture is long. Accordingly, it cannot be determined whether the posture is horizontal through the wearer's intention or the posture is horizontal through a fall. For example, when the wearer is sleeping in a horizontal posture, the safety confirmation system receives a signal representing that the tilt sensor has a horizontal posture over a long time, which should not be determined to be a fall. Accordingly, the method of detecting that the wearer wearing the tilt sensor has fallen on the basis of the output state of the tilt sensor is low in detection precision.

### SUMMARY

An advantage of some aspects of the invention is to solve at least a part of the problems described above and the invention can be embodied as the following forms or application examples.

### Application Example 1

According to this application example of the invention, there is provided a fall detecting device including: a detector unit that includes a plurality of tilt sensors each including a pair of electrodes disposed to oppose each other with the positional relationship therebetween being fixed and a conductor capable of freely moving between the pair of electrodes and changing an electrical-connection state between the pair of electrodes by means of the movement of the conductor resulting from a variation in posture of the pair of electrodes, wherein the plurality of tilt sensors are arranged so that directions in which the pairs of electrodes are opposed to each other are perpendicular to each other; a multivalued data output unit that acquires the electrical-connection states between the pair of electrodes of the plurality of tilt sensors and converts the acquired electrical-connection state into multivalued data on the basis of the ratios of the electrical-connection states in a predetermined first period; and a fall determining unit that determines the occurrence of a fall when a moving average value of the multivalued data of the plurality of tilt sensors in a second period is equal to or greater than a first threshold value, wherein the second period is an integral multiple of the first period.

According to this configuration, the fall determining unit is provided which determines the occurrence of a fall when the moving average value of the multivalued data in the second period of the respective tilt sensors is equal to or greater than the first threshold value. Accordingly, the time point when the ratio of the period in which the electrical-connection state between the pair of electrodes is an electrically-disconnected state in the first period is raised continuously in all the tilt sensors can be detected from the moving average value of the multivalued data. Since the posture of the detector unit rapidly varies at the time point when a wearer wearing the fall detecting device starts falling, there is high possibility that the conductors of all the tilt sensors of the detector unit move away from the pairs of electrodes into a non-contact state. Accordingly, by detecting the time point when the ratio of the period in which the electrical-connection state between the pair of electrodes in the first period is the electrically-disconnected state is raised continuously in all the tilt sensors from the moving average value of the multivalued data, it is possible to detect that the posture of the detector unit rapidly varies. As a result, it is possible to improve the detection precision for detecting that a wearer wearing the fall detecting device has fallen.

### Application Example 2

In this application example of the invention, the fall determining unit determines the occurrence of a fall when a period from a first time point to a second time point is equal to or less than a predetermined third period, where the first time point is when the moving average value of the multivalued data of the plurality of tilt sensors in the second period is equal to or greater than the first threshold value and the second time point is when the moving average value of the multivalued data in the second period of the tilt sensor in which the pair of electrodes is disposed to be opposed to the vertical direction before the variation in posture is equal to or less than a second threshold value.

According to this configuration, the condition of determining the occurrence of a fall when the period from the first time point to the second time point is equal to or less than the third period is added. Accordingly, when the period from the first time point to the second time point is greater than the third period, this is not determined to be a fall. As a result, it is possible to suppress the erroneous detection of detecting the occurrence of a fall when a fall does not occur but a rapid variation in posture is maintained for a long time and thus to further improve the detection precision for detecting a fall.

### Application Example 3

In this application example of the invention, each tilt sensor includes the pair of electrodes whose hemispherical concave faces are opposed to each other and the conductor having a spherical shape and the conductor moves in a spherical space formed by the hemispherical concave faces of the pair of electrodes due to the variation in posture of the pair of electrodes.

According to this configuration, the electrical-connection state between a pair of electrodes can be set to an electrically-connected state or an electrically-disconnected state depending on the variation in posture of the pair of electrodes.

### Application Example 4

According to this application example of the invention, there is provided a fall detecting method including: arranging a plurality of tilt sensors each including a pair of electrodes disposed to oppose each other with the positional relationship therebetween being fixed and a conductor capable of freely moving between the pair of electrodes and changing an electrical-connection state between the pair of electrodes by means of the movement of the conductor resulting from a variation in posture of the pair of electrodes so that directions in which the pairs of electrodes are opposed to each other are perpendicular to each other, acquiring the electrical-connection states between the pair of electrodes of the plurality of tilt sensors, and converting the acquired electrical-connection state into multivalued data on the basis of the ratios of the electrical-connection states in a predetermined first period; and determining the occurrence of a fall when a moving average value of the multivalued data of the plurality of tilt sensors in a second period is equal to or greater than a first threshold value, wherein the second period is an integral multiple of the first period.

According to this configuration, determining the occurrence of a fall when the moving average value of the multivalued data in the second period of the respective tilt sensors is equal to or greater than the first threshold value is provided. Accordingly, the time point when the ratio of the period in which the electrical-connection state between the pair of electrodes is an electrically-disconnected state in the first period is raised continuously in all the tilt sensors can be detected from the moving average value of the multivalued data. Since the posture of the detector unit rapidly varies at the time point when a wearer wearing the fall detecting device starts falling, there is high possibility that the conductors of all the tilt sensors of the detector unit move away from the pairs of electrodes into a non-contact state. Accordingly, by detecting the time point when the ratio of the period in which the electrical-connection state between the pair of electrodes in the first period is the electrically-disconnected state is raised continuously in all the tilt sensors from the moving average value of the multivalued data, it is possible to detect that the posture of the detector unit rapidly varies. As a result, it is possible to improve the detection precision for detecting that a wearer wearing the fall detecting device has fallen.

### Application Example 5

In this application example of the invention, the occurrence of a fall is determined when a period from a first time point to a second time point is equal to or less than a predetermined third period, where the first time point is when the moving average value of the multivalued data of the plurality of tilt sensors in the second period is equal to or greater than the first threshold value and the second time point is when the moving average value of the multivalued data in the second period of the tilt sensor in which the pair of electrodes is disposed to be opposed to the vertical direction before the variation in posture is equal to or less than a second threshold value.

According to this configuration, the occurrence of a fall is determined when the period from the first time point to the second time point is equal to or less than the third period is determined. Accordingly, when the period from the first time point to the second time point is greater than the third period, this is not determined to be a fall . As a result, it is possible to suppress the erroneous detection of detecting the occurrence of a fall when a fall does not occur but a rapid variation in posture is made for a long time and thus to further improve the detection precision for detecting a fall.

### BRIEF DESCRIPTION OF THE DRAWINGS

The invention will be described with reference to the accompanying drawings, wherein like numbers reference like elements.

FIG. 1 is a block diagram illustrating the configuration of a fall detecting device according to a first embodiment of the invention.

FIG. 2 is a perspective view illustrating the appearance of a tilt sensor.

FIG. 3 is a perspective view illustrating the appearance of tilt sensors of a detector unit having a normal posture.

FIG. 4 is a perspective view illustrating the appearance of the tilt sensors of the detector unit having a fallen posture.

FIG. 5 is a diagram illustrating rectangular waves generated from a multivalued data output unit.

FIGS. 6A to 6C are graphs illustrating multivalued data of the tilt sensors from the normal posture not falling to the fallen posture.

FIG. 7 is a flow diagram illustrating the process flow of a program according to the first embodiment of the invention.

FIGS. 8A to 8C are graphs illustrating multivalued data of the tilt sensors from the normal posture not falling to the fallen posture.

FIG. 9 is a flow diagram illustrating the process flow of a program according to a second embodiment of the invention.

FIG. 10A is a diagram illustrating a tilt sensor including a pair of electrodes whose curved faces are opposed to each other and a cylindrical conductor and FIG. 10B is a diagram illustrating a tilt sensor including a pair of electrodes whose curved faces are opposed to each other and a spherical conductor.

### DESCRIPTION OF EXEMPLARY EMBODIMENTS

### First Embodiment

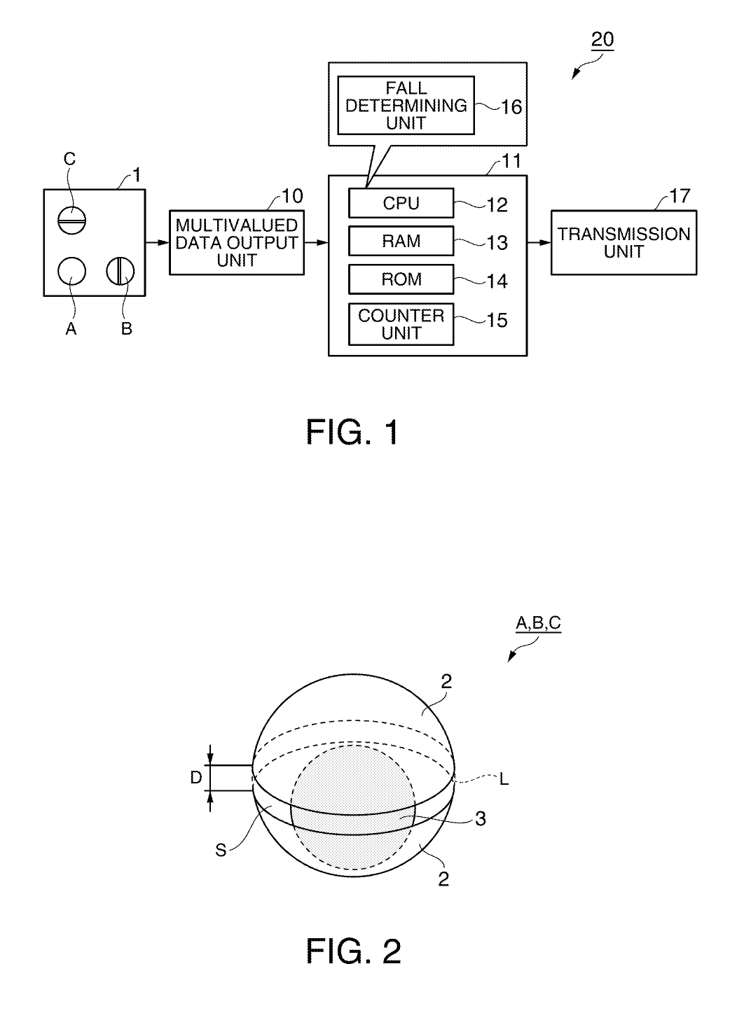

Hereinafter, a first embodiment of the invention will be described with reference to the accompanying drawings. FIG. 1 is a block diagram illustrating the configuration of a fall detecting device 20 according to the first embodiment.

A detector unit 1 includes tilt sensors A, B, and C. An electrical-connection state of each of tilt sensors A, B, and C is switched between an electrically-connected state (hereinafter, referred to as “ON” state) and an electrically-disconnected state (hereinafter, referred to as “OFF” state) due to a variation in posture of the detector unit 1.

A multivalued data output unit 10 shown in FIG. 1 is formed of an electronic circuit. The multivalued data output unit 10 acquires the electrical-connection state of tilt sensors A, B, and C of the detector unit 1 as two-valued data having two steps of ON and OFF, converts the electrical-connection state into multivalued data having four steps of 0, 1, 2, and 3, on the basis of the ratio of an ON or OFF period to a predetermined period, and outputs the multivalued data.

A control unit 11 shown in FIG. 1 includes a CPU 12, a RAM 13, a ROM 14, and a counter unit 15. The CPU 12 reads a program stored in the ROM 14 into the RAM 13 and executes the read program. The RAM 13 temporarily stores the multivalued data acquired from the multivalued data output unit 10. The CPU 12 can acquire the time from the counter unit 15.

A fall determining unit 16 detects that the detector unit 1 falls on the basis of the multivalued data stored in the RAM 13 and outputs a signal to a transmission unit 17. The fall determining unit 16 is formed of a program stored in the ROM 14 and is activated by causing the CPU 12 to read the program stored in the ROM 14 into the RAM 13 and to execute the read program.

The transmission unit 17 has a function of wirelessly sending a signal representing the fall to an external receiver (not shown). When acquiring the signal representing a fall from the fall determining unit 16, the transmission unit 17 wirelessly sends the signal representing a fall to the external receiver.

The detector unit 1 will be described in detail below.

FIG. 2 is a perspective view illustrating the appearance of tilt sensors A, B, and C of the detector unit 1. Each of tilt sensors A, B, and C includes a pair of electrodes 2 in which hemispherical concave faces are opposed to each other. The pair of electrodes 2 is supported by an insulating member (not shown) and is fixed in position with a gap D therebetween in a non-contact state.

The pair of electrodes 2 is formed using a method of plating the hemispherical concave faces formed of the insulating member with gold. Alternatively, a method of plastically processing a conductive material may be used.

By disposing the pair of electrodes 2 to oppose each other, a spherical space S is formed by the pair of hemispherical concave faces and a face, which is indicated by a broken line L in the gap D, as an extension of the concave faces. A spherical conductor 3 is disposed in the spherical space S . The conductor 3 moves in the space S, since the posture of the pair of electrodes 2 varies depending on the variation in posture of the detector unit 1. Accordingly, since the conductor 3 comes in contact with the pair of electrodes 2 at the same time, comes in contact with only one electrode 2, or comes in dis-contact with both electrodes 2 depending on the variation in posture of the pair of electrodes 2, the electrical-connection state between the pair of electrodes 2 varies.

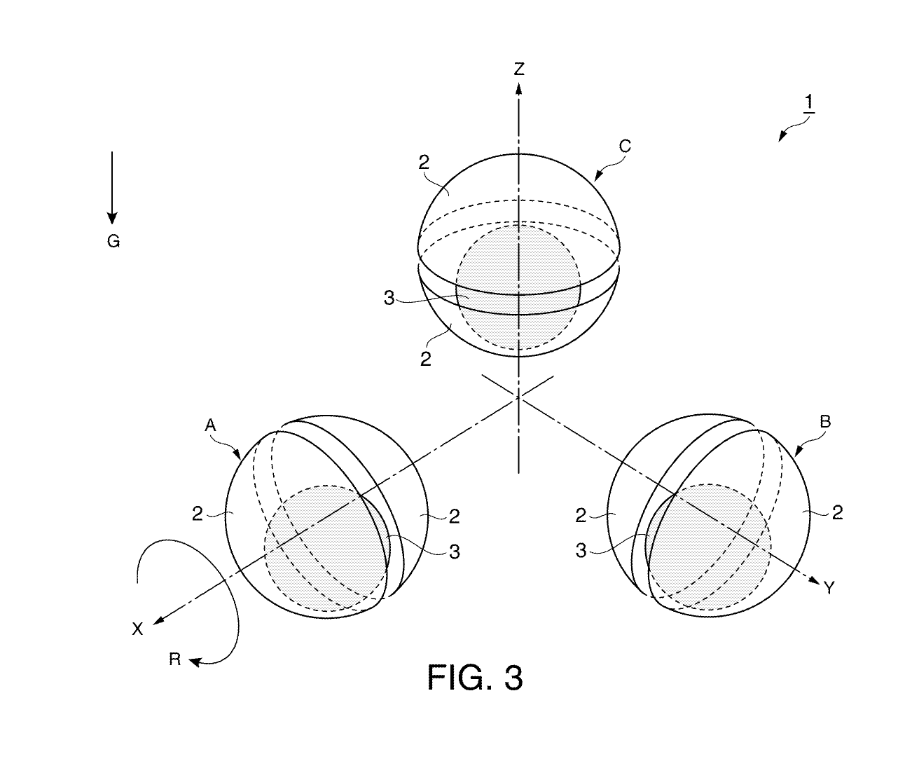

FIG. 3 is a perspective view illustrating the appearance of tilt sensors A, B, and C of the detector unit 1 with a normal posture. The Z axis in the vertical direction G is perpendicular to the horizontal plane formed of the X axis and the Y axis perpendicular to the X axis.

Tilt sensor A in which the concave faces of the pair of electrodes 2 are opposed in the X axis direction, tilt sensor B in which the concave faces of the pair of electrodes 2 are opposed in the Y axis direction, and tilt sensor C in which the concave faces of the pair of electrodes 2 are opposed in the Z axis direction are arranged in the detector unit 1 shown in FIG. 3.

When the detector unit 1 has the normal posture shown in FIG. 3, the conductors 3 of tilt sensor A and tilt sensor B come in contact with the pair of electrodes 2 and the electrical-connection state between the pair of electrodes 2 is the ON state. When the detector unit 1 has the normal posture shown in FIG. 3, the conductor 3 of tilt sensor C comes in contact with one electrode 2 but does not come in contact with the other electrode 2, whereby the electrical-connection state between the pair of electrodes 2 is the OFF state.

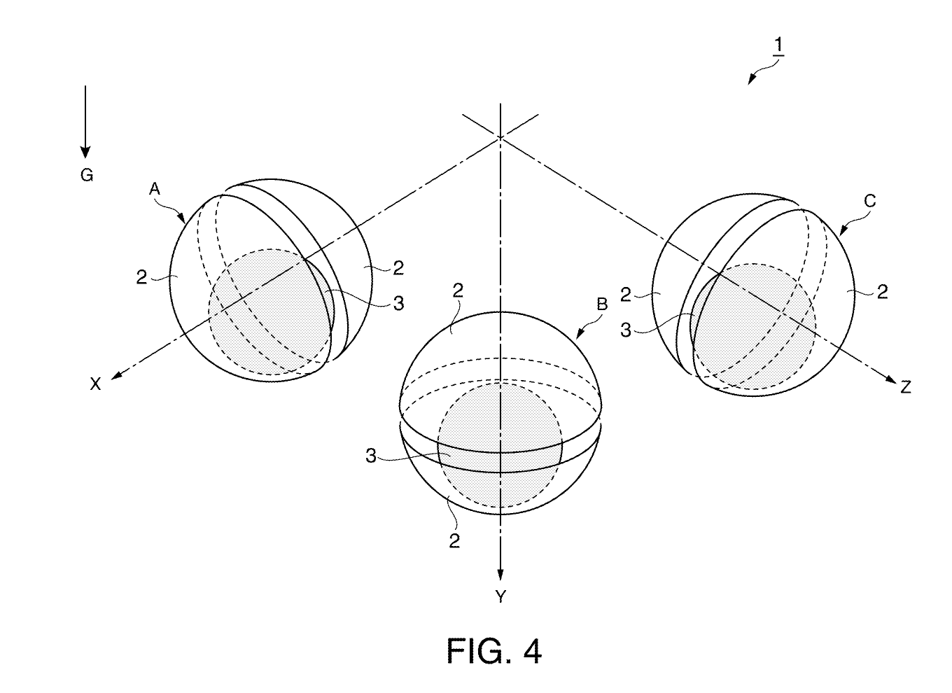

FIG. 4 is a perspective view illustrating the appearance of tilt sensors A, B, and C of the detector unit 1 having a fallen posture by rotating the detector unit 1 having the normal posture shown in FIG. 3 by 90 degrees in the rotation direction R about the X axis. When the detector unit 1 has the fallen posture shown in FIG. 4, the conductors 3 of tilt sensor A and tilt sensor C come in contact with the pair of electrodes 2, whereby the electrical-connection state between the pair of electrodes 2 is the ON state. The conductor 3 of tilt sensor B comes in contact with one electrode 2 but does not come in contact with the other electrode 2, whereby the electrical-connection state between the pair of electrodes 2 is OFF state.

The fall detecting device 20 is worn so that the detector unit 1 has the normal posture shown in FIG. 3 when a wearer wearing the fall detecting device 20 has a normal posture. Accordingly, when the wearer falls, the posture of the detector unit 1 is changed to the posture shown in FIG. 4.

A multivalued data output unit 10 will be described in detail below.

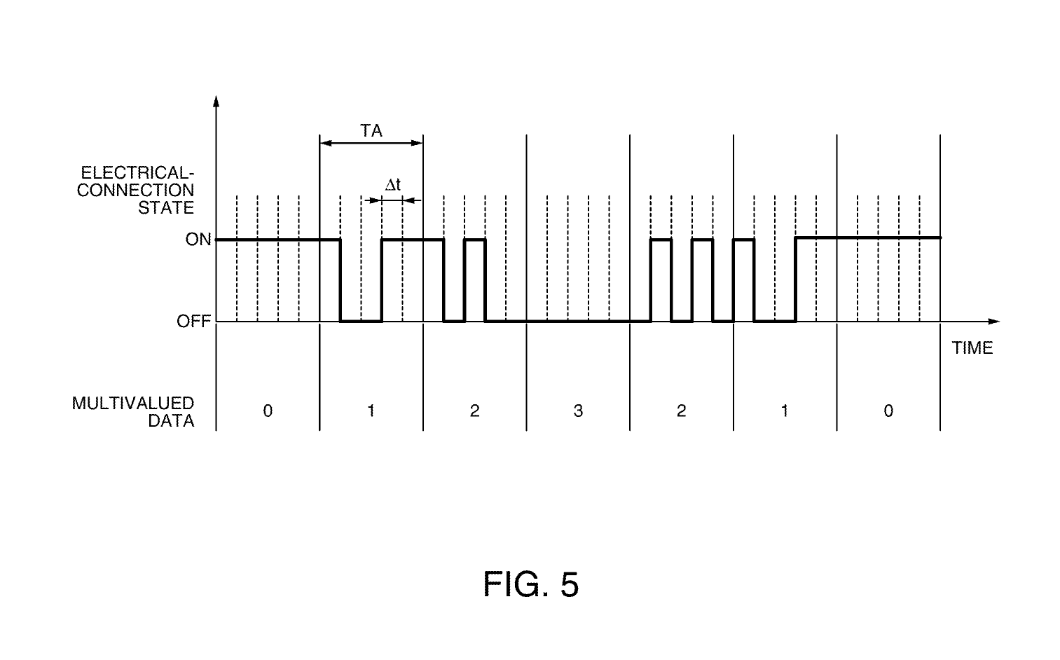

FIG. 5 shows a rectangular wave generated by the multivalued data output unit 10. The horizontal axis represents the time and the vertical axis represents the electrical-connection state of tilt sensor A by the use of ON or OFF. The multivalued data output unit 10 samples and detects the electrical-connection state between the pair of electrodes 2 of tilt sensor A every sampling time Δt. The sampling time Δt is set, for example, to 5 ms to 10 ms.

The multivalued data output unit 10 generates a rectangular wave so that the period Δt is ON when the sampled electrical-connection state between the pair of electrodes 2 is the ON state and the period Δt is OFF when the sampled electrical-connection state between the pair of electrodes 2 is the OFF state.

The multivalued data output unit 10 outputs the electrical-connection state between the pair of electrodes 2 as the multivalued data as the comparison result of the periods of ON and the periods of OFF in a predetermined first period TA. In this embodiment, the first period TA is set, for example, to a fifth multiple of the sampling time Δt. The multivalued data is set as follows from the electrical-connection state between the pair of electrodes 2.

When all the periods in the first period TA are ON, 0 is set as the multivalued data.

When total periods of ON≧total periods of OFF in the first period TA is satisfied, 1 is set as the multivalued data.

When total periods of ON<total periods of OFF in the first period TA is satisfied, 2 is set as the multivalued data.

When all the periods are OFF in the first period TA, 3 is set as the multivalued data.

The values of the multivalued data shown in the lower part of FIG. 5 are set as described above. The multivalued data output unit 10 samples the pair of electrodes 2 of tilt sensors B and C and sets the multivalued data, similarly to tilt sensor A.

As described above, the multivalued data output unit 10 acquires the electrical-connection state between the pair of electrodes 2 of tilt sensors A, B, and C as two-valued data having two steps of ON and OFF, and converts the electrical-connection state into multivalued data having four steps of 0, 1, 2, and 3 on the basis of the ratio of the ON or OFF periods to the first period TA.

A method of allowing the fall determining unit 16 to detect a fall from the multivalued data acquired from the multivalued data output unit 10 will be described below. In this embodiment, when a wearer wearing the fall detecting device 20 falls, it is assumed that the detector unit 1 with the normal posture shown in FIG. 3 rotates by 90 degrees in the rotation direction R about the X axis and is stopped with the fallen posture shown in FIG. 4.

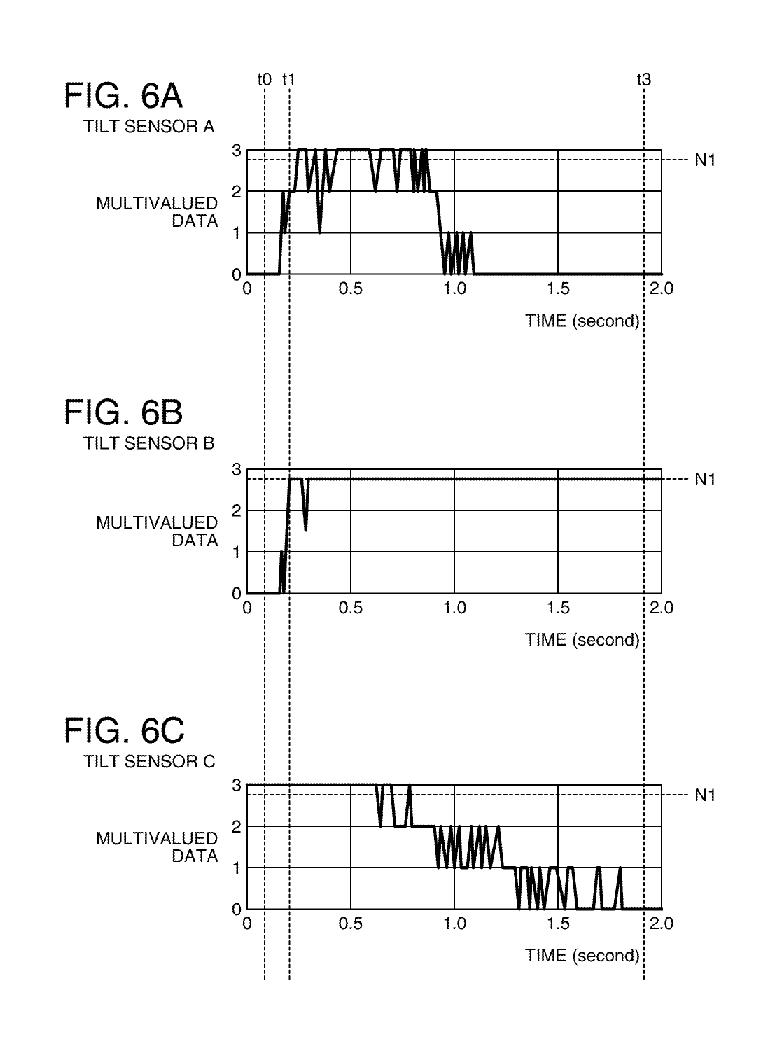

The multivalued data output unit 10 sequentially outputs the multivalued data described with reference to FIG. 5 to the control unit 11. FIG. 6A is a graph illustrating the multivalued data of tilt sensor A from the normal posture shown in FIG. 3 before a fall to the fallen posture shown in FIG. 4 after the fall. Similarly, FIG. 6B is a graph illustrating the multivalued data of tilt sensor B. Similarly, FIG. 6C is a graph illustrating the multivalued data of tilt sensor C. In FIGS. 6A to 6C, the horizontal axis represents the time (seconds) and the vertical axis represents the multivalued data.

In FIGS. 6A to 6C, at a time point t0 before the fall, the multivalued data of tilt sensor A and tilt sensor B shown in FIG. 3 is 0 since the entire first period TA is the period of ON, and the multivalued data of tilt sensor C is 3 since the entire first period TA is the period of OFF.

In FIGS. 6A to 6C, at a first time point t1 when a fall is started, since the conductors 3 of tilt sensor A and tilt sensor B roll along the concave faces or move apart from the concave faces, the ratio of the period of OFF to the first period TA in the electrical-connection state between the pair of electrodes 2 increases. Accordingly, the multivalued data is often 2 or 3. At the first time point t1 when the fall is started, since the electrical-connection state of tilt sensor C is the OFF state, the multivalued data is 3.

In this way, the possibility that the values of the multivalued data of tilt sensors A, B, and C are two or greater is high at the first time point t1.

Therefore, the fall determining unit 16 of the control unit 11 calculates the moving average value of immediate multivalued data in a predetermined second period, which are sequentially output from the multivalued data output unit 10, and determines the occurrence of a fall when the calculated moving average value is equal to or greater than a first threshold value N1.

The second period is set to an integral multiple of the first period TA shown in FIG. 5. For example, the second period is determined as 50-th multiples of the first period TA. The first threshold value N1 is experimentally determined and is, for example, 2.8.

In FIGS. 6A to 6C, at a time point t3 after the fall, the multivalued data of tilt sensor A and tilt sensor C of the detector unit 1 with the fallen posture shown in FIG. 4 is 0 since the entire first period TA is the period of ON. The multivalued data of tilt sensor B is 3 since the entire first period TA is the period of OFF.

In FIGS. 6A to 6C, in the period from the first time point t1 when the fall starts to the time point t3 after the fall, the multivalued data of tilt sensors A, B, and C is as shown in the graph of FIGS. 6A to 6C.

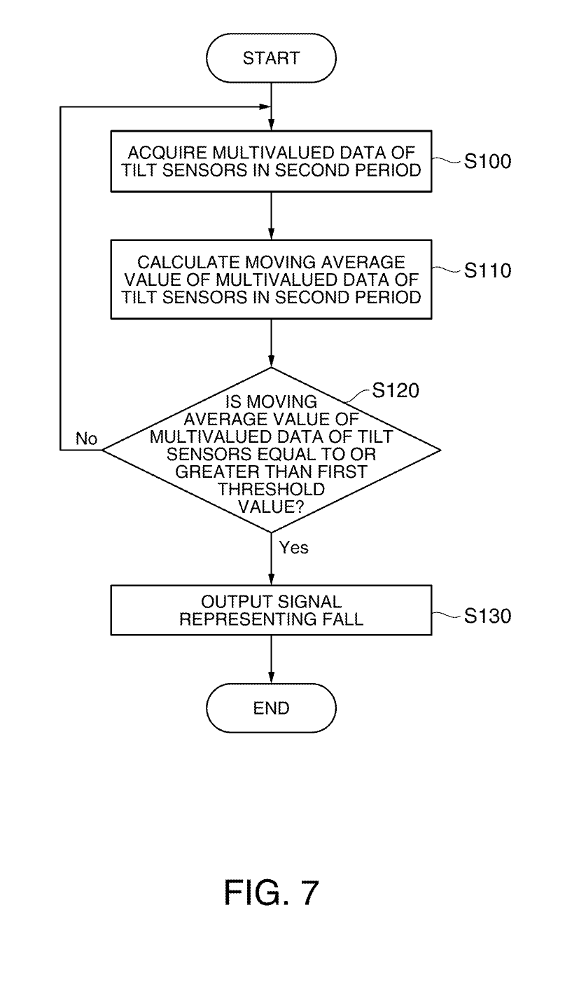

Processes of a program performed by the fall determining unit 16 will be described below. FIG. 7 is a flow diagram illustrating the process flow of the program according to this embodiment. In step S100, the fall determining unit 16 acquires immediate multivalued data in the second period.

In step S110, the fall determining unit 16 calculates the moving average value of the multivalued data in the second period from the acquired multivalued data.

In step S120, the fall determining unit 16 determines whether the calculated moving average value is equal to or greater than the first threshold value N1. When it is determined that the moving average value is equal to or greater than the first threshold value N1, the fall determining unit 16 outputs a signal representing a fall to the transmission unit 17 in step S130. When it is determined that the moving average value is less than the first threshold value N1, the fall determining unit 16 performs the process of step S100 again.

The fall detecting device 20 according to this embodiment includes: the detector unit 1 that includes tilt sensors A, B, and C each including a pair of electrodes 2 fixed in position so as to oppose each other and the conductor 3 capable of freely moving between the pair of electrodes 2 and changing the electrical-connection state between the pair of electrodes 2 by means of the movement of the conductor 3 resulting from the variation in posture of the pair of electrodes 2, wherein tilt sensors A, B, and C are arranged so that directions in which the pairs of electrodes 2 are opposed to each other are perpendicular to each other; the multivalued data output unit 10 that acquires the electrical-connection states between the pair of electrodes 2 of tilt sensors A, B, and C and converts the acquired electrical-connection state into the multivalued data on the basis of the ratios of the electrical-connection states in the first period TA; and the fall determining unit 16 that determines the occurrence of a fall when the moving average value of the multivalued data of tilt sensors A, B, and C in the second period is equal to or greater than the first threshold value N1, wherein the second period is an integral multiple of the first period TA.

According to this configuration, at the first time point t1 when the wearer wearing the fall detecting device 20 starts falling, since the posture of the detector unit 1 rapidly varies, the possibility that the conductors 3 of tilt sensors A, B, and C of the detector unit 1 go apart from and do not come in contact with the pairs of electrodes 2 is high. Accordingly, by detecting a time point when the ratio of the period in which the electrical-connection state between the pair of electrodes 2 is the OFF state to the first period TA is continuously raised in tilt sensors A, B, and C from the moving average value of the multivalued data, it is possible to detect that the posture of the detector unit 1 rapidly varies. Therefore, it is possible to improve the detection precision for detecting that the wearer wearing the fall detecting device 20 falls.

Each of tilt sensors A, B, and C includes a pair of electrodes 2 in which the hemispherical concave faces are opposed and a spherical conductor 3. The conductor 3 moves in a spherical space S formed by a pair of hemispherical concave faces depending on the variation in posture of the pair of electrodes 2.

According to this configuration, the electrical-connection state between the pair of electrodes 2 can be switched between ON and OFF depending on the variation in posture of the pair of electrodes 2.

The fall detecting method according to this embodiment includes: a multivalued data output step of arranging tilt sensors A, B, and C, each of which includes the pair of electrodes 2 disposed to oppose each other with the positional relationship therebetween being fixed and the conductor 3 capable of freely moving between the pair of electrodes 2 and changes the electrical-connection state between the pair of electrodes 2 by means of the movement of the conductor 3 resulting from the variation in posture of the pair of electrodes 2, so that directions in which the pairs of electrodes 2 are opposed to each other are perpendicular to each other, acquiring the electrical-connection states between the pair of electrodes 2 of tilt sensors A, B, and C, and converting the acquired electrical-connection state into the multivalued data on the basis of the ratios of the electrical-connection states in the first period TA; and a fall determining step of determining the occurrence of a fall when the moving average value of the multivalued data of tilt sensors A, B, and C in the second period is equal to or greater than the first threshold value N1, wherein the second period is an integral multiple of the first period TA.

### Second Embodiment

In a second embodiment of the invention, a method of suppressing an erroneous detection will be described by excluding a case where a wearer wearing the fall detecting device has not fallen but rapidly moves for a long time using multivalued data of the electrical-connection state of tilt sensor C in which the opposing direction of the pair of electrodes 2 is parallel to the vertical direction G shown in FIG. 3 before the fall.

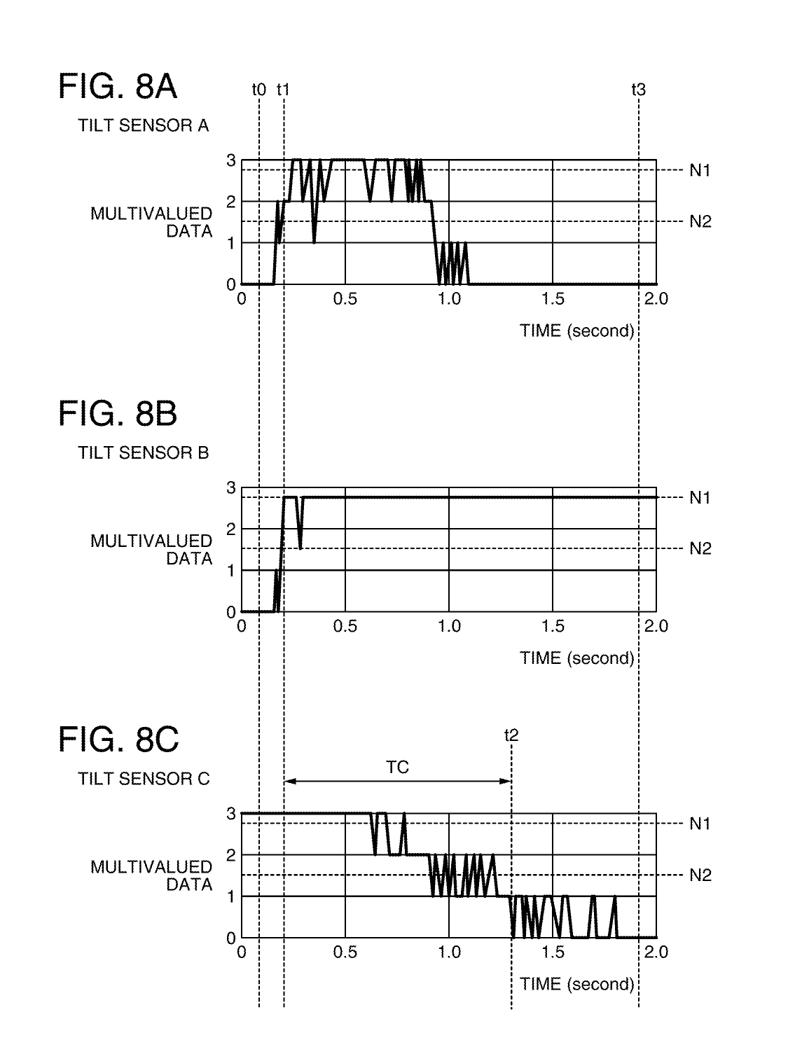

The graphs shown in FIGS. 8A to 8C are the same as the graphs shown in FIGS. 6A to 6C according to the first embodiment and illustrate the multivalued data of tilt sensors A, B, and C from the normal posture shown in FIG. 3 before the fall to the fallen posture shown in FIG. 4.

A second time point t2 in FIG. 8C represents a time point when the moving average value of the multivalued data of tilt sensor C in the second period is equal to or less than a second threshold value N2. The second threshold value N2 is experimentally determined and is, for example, 1.5.

In the second embodiment, the fall determining unit 16 detects the second time point t2. After the second time point t2, the multivalued data of tilt sensor C is alternately 1 or 0. At the time point t3 after the fall, the multivalued data is 0. The fall determining unit 16 calculates a period TC from the first time point t1, which has been described with reference to FIGS. 6A to 6C in the first embodiment, to the second time point t2.

The fall determining unit 16 detects the occurrence of a fall when the moving average value of the multivalued data of tilt sensors A, B, and C in the second period is equal to or greater than the first threshold value N1 and when the period TC from the first time point t1 to the second time point t2 is equal to or less than a predetermined third period. The third period for comparison is experimentally determined and is, for example, 1.5 seconds.

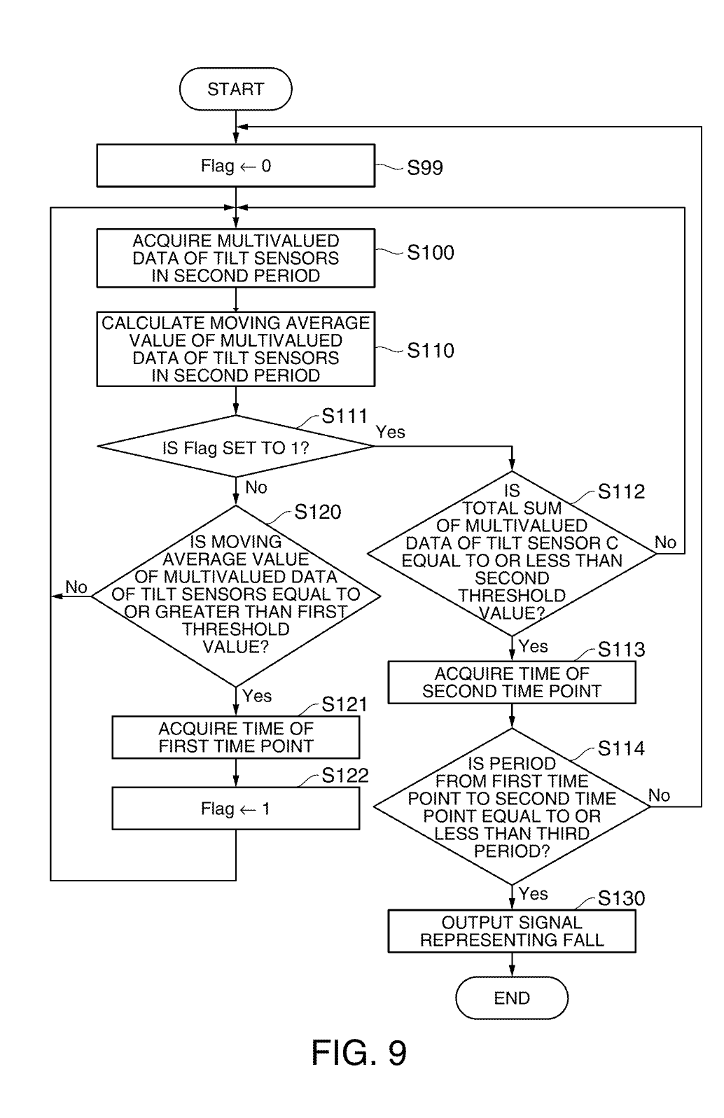

Processes of a program performed by the fall determining unit 16 in the second embodiment will be described below. FIG. 9 is a flow diagram illustrating the process flow of the program according to the second embodiment. In FIG. 9, steps S99, S111, S121, S122, and S112 to S114 are added to the flow diagram shown in FIG. 7 in the first embodiment.

In step S99, the fall determining unit 16 sets Flag to 0. In step S100, the fall determining unit 16 acquires immediate multivalued data in the second period.

In step S110, the fall determining unit 16 calculates the moving average value of the multivalued data in the second period from the acquired multivalued data.

In step S111, the fall determining unit 16 determines whether Flag is 1. When it is determined that Flag is 1 (YES), the process of step S112 is performed. When it is determined that Flag is not 1 (NO), the process of step S120 is performed. Here, since Flag is not 0, the process of step S120 is performed.

In step S120, the fall determining unit 16 determines whether the calculated moving average value is equal to or greater than the first threshold value N1. When it is determined that the moving average value is equal to or greater than the first threshold value N1 (YES), the fall determining unit 16 performs the process of step S121. When it is determined that the moving average value is less than the first threshold value N1 (NO), the fall determining unit 16 performs the process of step S100 again. Here, since the moving average value is equal to or greater than the first threshold value N1, the fall determining unit 16 performs the process of step S121.

In step S121, the fall determining unit 16 acquires the time of the first time point t1 from the counter unit 15. In step S122, the fall determining unit 16 sets Flag to 1 and performs the process of step S100 again.

In steps S100 and S110, the fall determining unit 16 performs the processes as described above. In step S111, the fall determining unit 16 determines whether Flag is 1. Here, since Flag is 1, the fall determining unit 16 performs the process of step S112.

In step S112, the fall determining unit 16 determines whether the total sum of the multivalued data of tilt sensor C in the second period is equal to or less than the second threshold value N2. The fall determining unit 16 performs the process of step S113 when it is determined that the total sum of the multivalued data is equal to or less than the second threshold value N2 (YES) and performs the process of step S100 again when the total sum of the multivalued data is greater than the second threshold value N2.

In step S113, the fall determining unit 16 acquires the time of the second time point t2 from the counter unit 15. In step S114, the fall determining unit 16 determines whether the period TC (see FIG. 8C) from the first time point t1 to the second time point t2 is equal to or less than the third period. The process of step S130 is performed when it is determined that the period is equal to or less than the third period (YES). The process of step S99 is performed again when it is determined that the period is greater than the third period (NO). In step S130, the fall determining unit 16 outputs a signal representing a fall to the transmission unit 17.

The other configuration of the fall detecting device described in this embodiment is the same as the fall detecting device 20 described in the first embodiment.

In the fall detecting device according to this embodiment, the fall determining unit 16 determines the occurrence of a fall when the condition that the moving average value of the multivalued data of tilt sensors A, B, and C in the second period is equal to or greater than the first threshold value N1 and the condition that the period TC from the first time point t1 to the second time point t2 is equal to or less than the third period are satisfied, where the first time point t1 is when the moving average value of the multivalued data of tilt sensors A, B, and C in the second period is equal to or greater than the first threshold value N1 and the second time point t2 is when the moving average value of the multivalued data in the second period of tilt sensor C in which the pair of electrodes 2 is disposed to be opposed to the vertical direction G before the variation in posture is equal to or less than the second threshold value N2.

According to this configuration, when the period TC from the first time point t1 to the second time point t2 is greater than the third period, this is not determined to be a fall. Accordingly, it is possible to suppress the erroneous detection of erroneously detecting the occurrence of a fall when a rapid variation in posture is made for a long time instead of the fall, thereby improving the detection precision for detecting a fall.

In the fall detecting method according to this embodiment, a fall determining step includes determining the occurrence of a fall when the condition that the moving average value of the multivalued data of tilt sensors A, B, and C in the second period is equal to or greater than the first threshold value N1 and the condition that the period TC from the first time point t1 to the second time point t2 is equal to or less than the third period are satisfied, where the first time point t1 is when the moving average value of the multivalued data of tilt sensors A, B, and C in the second period is equal to or greater than the first threshold value N1 and the second time point t2 is when the moving average value of the multivalued data in the second period of tilt sensor C in which the pair of electrodes 2 is disposed to be opposed to the vertical direction G before the variation in posture is equal to or less than the second threshold value N2.

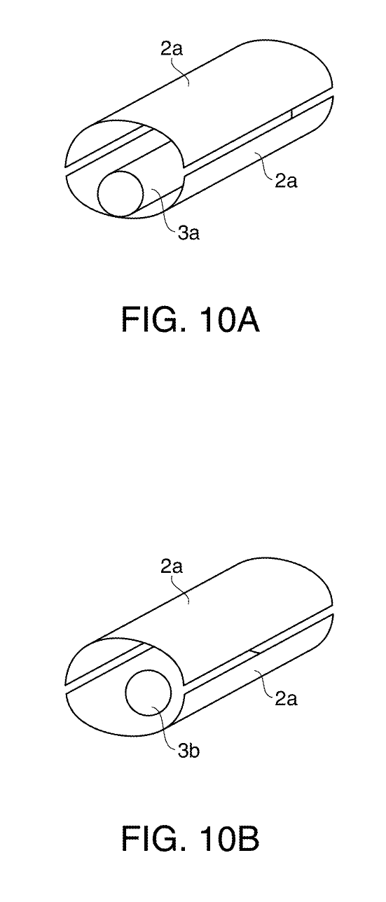

In the first and second embodiments, tilt sensors A, B, and C each including a pair of electrodes 2 having hemispherical concave faces and a conductor 3 are employed. However, as shown in FIG. 10A, a tilt sensor including a pair of electrodes 2a having curved faces opposed to each other and a cylindrical conductor 3a may be employed. As shown in FIG. 10B, a tilt sensor including a pair of electrodes 2a having curved faces opposed to each other and a spherical conductor 3b may be employed.

The entire disclosure of Japanese Patent Application No. 2010-050232, filed Mar. 8, 2010 is expressly incorporated by reference herein.

What is claimed is:

1. A fall detecting device comprising:
a detector unit that includes a plurality of tilt sensors each including a pair of electrodes disposed to oppose each other with the positional relationship therebetween being fixed and a conductor capable of freely moving between the pair of electrodes and changing an electrical-connection state between the pair of electrodes by means of the movement of the conductor resulting from a variation in posture of the pair of electrodes, wherein the plurality of tilt sensors are arranged so that directions in which the pairs of electrodes are opposed to each other are perpendicular to each other;
a multivalued data output unit that acquires the electrical-connection states between the pair of electrodes of the plurality of tilt sensors and converts the acquired electrical-connection state into multivalued data on the basis of the ratios of the electrical-connection states in a predetermined first period; and
a fall determining unit that determines the occurrence of a fall when a moving average value of the multivalued data of the plurality of tilt sensors in a second period is equal to or greater than a first threshold value, wherein the second period is an integral multiple of the first period.

2. The fall detecting device according to claim 1, wherein the fall determining unit determines the occurrence of a fall when a period from a first time point to a second time point is equal to or less than a predetermined third period, where the first time point is when the moving average value of the multivalued data of the plurality of tilt sensors in the second period is equal to or greater than the first threshold value and the second time point is when the moving average value of the multivalued data in the second period of the tilt sensor in which the pair of electrodes is disposed to be opposed to the vertical direction before the variation in posture is equal to or less than a second threshold value.

3. The fall detecting device according to claim 1, wherein each tilt sensor includes the pair of electrodes whose hemispherical concave faces are opposed to each other and the conductor having a spherical shape and the conductor moves in a spherical space formed by the hemispherical concave faces of the pair of electrodes due to the variation in posture of the pair of electrodes.

4. A fall detecting method comprising:
arranging a plurality of tilt sensors each including a pair of electrodes disposed to oppose each other with the positional relationship therebetween being fixed and a conductor capable of freely moving between the pair of electrodes and changing an electrical-connection state between the pair of electrodes by means of the movement of the conductor resulting from a variation in posture of the pair of electrodes so that directions in which the pairs of electrodes are opposed to each other are perpendicular to each other, acquiring the electrical-connection states between the pair of electrodes of the plurality of tilt sensors, and converting the acquired electrical-connection state into multivalued data on the basis of the ratios of the electrical-connection states in a predetermined first period; and
determining the occurrence of a fall when a moving average value of the multivalued data of the plurality of tilt sensors in a second period is equal to or greater than a first threshold value, wherein the second period is an integral multiple of the first period.

5. The fall detecting method according to claim 4, wherein the occurrence of a fall is determined when a period from a first time point to a second time point is equal to or less than a predetermined third period is determined, where the first time point is when the moving average value of the multivalued data of the plurality of tilt sensors in the second period is equal to or greater than the first threshold value and the second time point is when the moving average value of the multivalued data in the second period of the tilt sensor in which the pair of electrodes is disposed to be opposed to the vertical direction before the variation in posture is equal to or less than a second threshold value.
